(19) United States
(12) Patent Application Publication
(10) Pub. No.: US 2016/0347695 A1
(43) Pub. Date: Dec. 1, 2016

(54) APPARATUS AND METHODS FOR METHANOL GENERATION

(71) Applicant: SOLOMON ZAROMB, BURR RIDGE, IL (US)

(72) Inventor: SOLOMON ZAROMB, BURR RIDGE, IL (US)

(21) Appl. No.: 14/721,856

(22) Filed: May 26, 2015

(51) Int. Cl. C07C 29/48; C10L 1/02; B01J 19/24

(52) U.S. Cl. CPC C07C 29/48; B01J 19/24; C10L 1/02; B01J 2219/24; C10L 2290/10

(57) ABSTRACT

To alleviate global warming while providing a liquid fuel to replace gasoline, apparatus and methods are disclosed for generating methanol from natural gas via the syngas route. Said apparatus and methods can also serve to dispose of any CO₂ that is captured from fuel burning exhausts, especially from motor vehicle exhausts. With a proper choice of reagent ratios, the energy required for the methanol generation may derive from exothermic reactions yielding enough heat to make up for the endothermic reaction energy involved in steam reforming of natural gas, thus obviating the need for any substantial electric energy input. Although steam reforming of natural gas may yield a methanol-rich fuel that will outcompete gasoline by a wide margin, it would still fail by itself to significantly alleviate global warming. However, inclusion of captured CO₂ in the proper ratio in the syngas reaction may yield an inexpensive conversion of said CO₂ and natural gas to methanol or its derivatives without calling for any significant electric energy consumption. The preferred ratio is 1 mole of CO₂ to 2 moles of H₂O to 3 moles of CH₄. These reagents may be introduced into a methanol synthesis reactor preferably maintained at about 250-300 C and 50-100 atm comprising a Cu—ZnO-alumina catalyst or its equivalent.

### REFERENCE TO RELATED APPLICATIONS

This is a non-provisional application of provisional application No. 61/997,157, filed May 27, 2014. This application is related to my U.S. Pat. No. 8,413,420, the disclosures of which are incorporated herein by reference.

### BACKGROUND OF THE INVENTION

The afore-cited U.S. Pat. No. 8,413,420 discloses apparatus and methods for capturing CO₂ from motor vehicle exhausts and converting it into methanol and other useful products.

As envisaged in said patent, the captured CO₂ is converted to methanol or its derivatives or by-products by either reacting it with electrolytically generated hydrogen or by operating a water and CO₂ consuming reverse fuel cell. Either method would entail a considerable outlay of electrical energy which would add to its cost and indirectly to its carbon imprint. It is therefore a prime object of my invention to provide a means of converting captured CO₂ to methanol or its derivatives without significant electrical energy input or otherwise increased carbon imprint.

This invention relates to systems and methods for economically generating methanol from natural gas and carbon dioxide, so as to yield an inexpensive greenhouse-friendly methanol fuel that would be highly competitive with gasoline and thereby help alleviate global warming and the impending exhaustion of worldwide supplies of petroleum.

The possibly disastrous consequences of global warming due to increasing atmospheric concentrations of carbon dioxide emanating from the burning of fossil fuels have been widely recognized in recent years, and my afore-cited U.S. Pat. No. 8,413,420 is aimed at greatly alleviating such warming by capturing CO₂ from motor vehicle exhausts and converting it to methanol fuel and other useful methanol products or derivatives. However, that conversion would require substantial amounts of electrical energy for either generating the needed hydrogen reagent by electrolysis or for the reversed fuel cell proposed in said patent. It is the purpose of this invention to avoid the need for hydrogen reagent or for a reversed fuel cell by generating methanol from methane and carbon dioxide via a “syngas reaction” route, as disclosed below.

It is an object of my invention to provide a low-cost energy-saving method of converting into methanol the CO₂ that may be captured not only from motor vehicle exhausts but also from flue gas streams produced by existing coal-fired electricity generating power plants and from other major CO₂-generating sources, such as cement production, oil and gas processing, petrochemical industry, iron and steel industry, or aluminum and other metals producers, so as to help alleviate global warming.

Another purpose of my invention is to delay the impending exhaustion of petroleum supplies by providing for inexpensive mass production of methanol from natural gas via the syngas route which may lead to the displacement of gasoline by methanol or its derivatives as the main automotive fuel and to reductions in global CO₂ emissions.

My invention also addresses the problem of the impending exhaustion of worldwide supplies of petroleum. In 1970, Hubbert of the U.S. Geological Survey made his famous assessent of the amount of available global oil resources and estimated that it would climax at about 2000. According to recent DOE publications, the estimated climax will most likely occur in the year 2016 or at the latest in 2037, after which time impending shortages of petroleum supplies would most adversely impact most forms of transportation and most global economies.

It is therefore a further object of my invention to provide a process for generating ample supplies of an alternative relatively inexpensive liquid fuel.

It is a still further object of my invention to effectuate major cost and energy savings by eliminating the need to compress captured CO₂ to pipeline pressure by converting it instead at a substantially lower pressure directly into a usable fuel, such as methanol.

It is also a further object of my invention to effectuate major cost and energy savings by dispensing with the need to transport captured CO₂ to a separate facility.

More objects of the invention will become apparent to professionals in the chemical and electrical engineering, metallurgical, environmental, law enforcement, and related areas following perusal of the complete specification.

### SUMMARY OF THE INVENTION

My invention aims to alleviate global warming by providing a cost-saving more greenhouse-friendly liquid fuel to replace gasoline and also disposing of captured carbon dioxide while generating said fuel.

This invention relates to systems and methods for economically generating methanol from natural gas and carbon dioxide, so as to yield an inexpensive more greenhouse-friendly methanol fuel that would be highly competitive with gasoline and thereby alleviate global warming and the impending exhaustion of worldwide supplies of petroleum.

My invention provides a low-cost energy saving method of converting into methanol or its derivatives the CO₂ that may be captured not only from motor vehicle exhausts but also from the flue gas streams produced by coal-fired electricity generating power plants and from other major CO₂-generating sources. My invention should reduce the need for sequestration of CO₂ and thereby effectuate major cost and energy savings by obviating the need to transport captured CO₂ to a separate facility and to compress captured CO₂ to pipeline pressure, by converting it instead at a substantially lower pressure directly into a usable fuel, such as methanol or its derivatives.

Briefly, my invention consists of providing for inexpensive mass production of methanol from natural gas via the syngas route which may lead to the displacement of gasoline by methanol as the main automotive fuel and to considerable reductions in global CO₂ emissions.

The recent and expected continued fall in natural gas prices opens the possibility of highly reduced costs of methanol production from steam-reformed natural gas via the reactions

CH₄+2H₂O→4H₂+CO₂ [1]

and

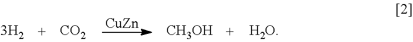

Although reactions 1 and 2 would not result in any net removal of CO₂, their implementation will yield a methanol fuel that will be more competitive than gasoline at current prices by a wide margin.

Equation 1 results from steam reforming of natural gas

CH₄+H₂O→CO+3H₂ [3]

followed by the water gas shift reaction

CO+H₂O→CO₂+H₂ [4]

However, of key interest is the reaction

CO+2H₂→CH₃OH [5].

Reactions 2, 3, and 5 when combined in the ratio of 1:3:3 yield substantially pure methanol

3CH₄+2H₂O+CO₂→4CH₃OH [6].

Equation 6 implies no need for significant electric power input to generate methanol from natural gas and captured CO₂. It also implies that captured CO₂ can also be converted to methanol in the same process without need of much additional electric power input.

### BRIEF DESCRIPTION OF THE DRAWING

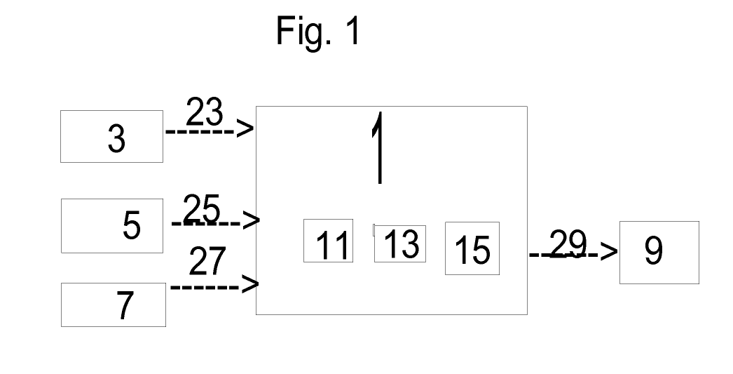

The invention is best explained with reference to the drawing, in which FIG. 1 is a block diagram of the key elements of my invention.

In the block diagram of FIG. 1, a methanol synthesis reactor 1, maintained by a temperature control system 11 preferably at about 250-300 C and by a pressure control system 13, preferably at about 50-100 atmospheres, is provided with a Cu—ZnO-alumina catalyst 15 or its equivalent for catalyzing the above reactions 3, 5, and 2. The reactor is fed from a supply 3 of methane or natural gas [CH₄] through a control valve 23, a supply 5 of steam [H₂O] through a valve 25, and a supply 7 of [CO₂] through a valve 27, so as to yield the overall reaction 6 when the reactants are introduced in the molar ratios of about 1CO₂ to 2H₂O to 3CH₄. The overall reaction product, CH₃OH, is fed to a methanol collector 9 through a valve 29, whence it is distributed for appropriate uses.

The reason for the feasibility of reaction 6 is that of reactions 2, 3, and 5, only the steam reforming reaction 3 is endothermic, requiring a heat input of about 250 kJ/Mol of CH₄. That heat might be supplied by the exothermic reactions 2¹ and 5² by having all three reactions set to run together in an optimal ratio in the methanol synthesis reactor 1. This could result in up to a mole of captured CO₂ being used up per 4 moles of generated CH₃OH. ¹ΔH≈130 kJ/Mol CH₃OH²ΔH≈128 kJ/Mol CH₃OH

By combining reactions 2, 3, and 5 in the ratio of 1:3:3 so as to yield substantially pure methanol

3CH₄+2H₂O+CO₂→4CH₃OH [6],

the need for signficant electric power input to generate methanol from natural gas and captured CO₂ is removed, which also implies that captured CO₂ can also be converted to methanol in the same process without need of much additional electric power input.

My recently issued U.S. Pat. No. 8,413,420 opens the way to the capture of CO₂ from motor vehicle exhausts and its conversion to methanol, possibly in repetitive cycles, thereby minimizing their atmospheric CO₂ imprint and thus alleviating global warming.

As envisaged in said patent, the captured CO₂ would be converted to methanol with a considerable electrical energy input. However, the recent and expected continued fall in natural gas prices opens the possibility of highly reduced costs of methanol production from steam-reformed natural gas via the above reactions 1 and 2. Although reactions 1 and 2 would not result in any net removal of CO₂, their implementation will yield a methanol fuel that will be more competitive than gasoline at current prices by a wide margin.

Indeed, estimates of <6 $/GJ for methanol from steam reformed gas translate to less than 0.9 $/gallon, as compared with recent methanol and gasoline prices of about 3 $/gallon.

Therefore, if generation of low-priced methanol be of foremost priority even without removal of any captured CO₂, an alternative embodiment of my invention could supply the heat required for the endothermic reaction 3 by burning some natural gas

CH₄+2O₂→2H₂O+CO₂ [7]

or some methanol

CH₃OH+1.5O₂→2H₂O+CO₂ [8].

Substitution of reaction 7 or 8 for reaction 2 would then yield a net excess of CO₂ contributing to global warming, but would still help replace gasoline by cleaner burning methanol and slow down the impending exhaustion of petroleum reserves.

The following steps and modifications are required for successful implementation of reactions 2, 3, and 5.

As noted above, of reactions 2, 3, and 5, only the steam reforming reaction 3 is endothermic, requiring a heat input of about 250 kJ/Mol of CH₄. That heat might be supplied by the exothermic reactions 2³ and 5⁴ by having all three reactions set to run together in an optimal ratio in the methanol synthesis reactor 1. This could result in up to a mole of captured CO₂ being converted per 4 moles of generated CH₃OH.

Much of the vast amount of research on optimizing the catalysts and temperature cycling for the most practical utilization of the preceding reactions has been reviewed by Song and Pan⁵ and more recently in a paper by Olah et al.⁶, both of which publications, included herein by reference, point to the care needed to avoid rapid deterioration of catalysts. Moreover, Olah et al. have arrived at reactant ratios similar to those of reaction 6 by combining steam reforming (my reaction 3) with the dry reforming reaction

CH₄+CO₂→2CO₊2H₂ [9].

With the availability of large supplies of natural gas at reduced prices, the possibility arises of mass producing low cost methanol via the syngas route and by reduced costs of ³ΔH 130 kJ/Mol CH₃OH⁴ΔH 128 kJ/Mol CH₃OH⁵Ch. Song, W. Pan, Tri-reforming of methane: a novel concept for catalytic production of industrially useful synthesis gas with desired H₂/CO ratios, Catatlysis Today 98 (2004) 463-484.⁶G. A, Olah, A, Goeppert, G. K. S. Prakash, Chemical Recycling of Carbon Dioxide to Methanol and Dimethyl Ether: From Greenhouse Gas to Renewable, Environmentally Carbon Neutral Fuels and Synthetic Hydrocarbons, J. Org. Chem. 74 (2009) 487-498.converting captured CO₂ to methanol, thereby displacing gasoline by the environmentally friendlier cleaner burning mehanol as the main automotive fuel.

Natural gas prices have dropped by more than 25% in recent times and are expected to stay that way for at least twenty or thirty years. The U.S. gas supplies are enormous now and sufficient for exporting natural gas for the next few decades. This points to the advantages of the steam reforming and methanol synthesis reactions 3 and 5. Hence the product of natural gas steam reformed is pretty much what is needed for synthesizing methanol. Of course, there would have to be a pressure change and a small amount of heat up to 300° C., whereupon the reaction occurs, perhaps with a further increase of 50° C. Now the point to note is cost. For the present practice, with the hydrogen obtained from water by electrolysis, our estimates show costs between $20 and $30/GJ. With the desired methanol made from natural gas, our estimated cost amounts to <$6/GJ including the heating and the pressurization. This shows a fourfold cost reduction when comparing <$6/GJ for methanol from natural gas with $25/GJ for methanol obtained with electrolysis. Of course there is a penalty to be paid for such an enormous price decrease in that reactions 1 and 2 would not remove CO₂ from the atmosphere and therefore would not alleviate global warming. Nevertheless, methanol in itself is a very worthy fuel [see Olah et al., footnote 6].

There will now be obvious many variations and modifications of the afore-disclosed embodiments to persons skilled in the art. It will be obvious that similar approaches can apply to the capture and conversion of CO.sub.2 from the smoke stacks of residential and office buildings, of various small industrial facilities, and other distributed small scale fuel burning sources. All these variations will fall within the scope of my invention if covered by the following claims.

1. Apparatus for inexpensive conversion of carbon dioxide from motor vehicle exhausts and other sources which comprises means for feeding said carbon dioxide to a methanol synthesis reactor, said reactor being maintained at a suitable temperature and pressure and comprising a catalyst for reacting each mole of said carhon dioxide with about 3 moles of methane and two moles of steam so as to yield methanol or a methanol derivative.

2. Apparatus of claim 1 wherein said carbon dioxide, methane, and steam are fed into said reactor in an approximate molar ratio of 1 to 3 to 2.

3. Apparatus of claim 1 wherein said carbon dioxide, methane, and steam are fed into said reactor through appropriate control valves.

4. Apparatus of claim 2 wherein said methanol or methanol derivative is fed from said reactor through appropriate valve controls to a methanol collector.

5. Apparatus for capturing carbon dioxide from motor vehicle exhausts and converting it to methanol or a derivative thereof at minimal cost which comprises: means for installing a cartridge loaded with a reversible CO₂-capturing agent in a motor vehicle and connecting it to the outlet of the vehicle's combustion source so as to cause the effluent of said source to pass through said cartridge; means for removing a CO₂-enriched cartridge from said vehicle and replacing it by a fresh cartridge; means for treating said CO₂-enriched cartridge so as to yield a stream of concentrated carbon dioxide, means for feeding said stream of carbon dioxide into a methanol synthesis reactor; means for supplying methane and steam to said reactor; and means for reacting said methane and steam with said stream of carbon dioxide in said reactor so as to form a chemical agent for powering a motor vehicle and for other uses.

6. Apparatus of claim 5, wherein said chemical agent contains methanol or its derivative.

7. An inexpensive method of converting carbon dioxide from motor vehicle exhausts and other sources which comprises the steps of feeding said carbon dioxide to a methanol synthesis reactor, maintaining said reactor at a suitable temperature and pressure, and reacting said carbon dioxide with methane and steam over a suitable catalyst so as to yield methanol or a derivative thereof.

8. The method of claim 7, which comprises the steps of: passing a carbon dioxide-containing air mixture emanating from a motor vehicle exhaust through a removable cartridge loaded with a reversible CO₂-capturing agent; generating a concentrated stream of carbon dioxide by heating said reversible CO₂-capturing agent; wherein said CO₂-capturing agent within said removable cartridge is connected to said motor vehicle exhaust; and providing a refueling station wherein a CO₂-enriched cartridge can be disconnected from said exhaust and replaced by a CO₂-depleted cartridge, and wherein the CO₂ from said enriched cartridge can be collected and converted into a methanol-containing fuel or other usable product.

9. The method of claim 8 wherein said effluent derives from the burning of a methanol-containing fuel.

10. The method of claim 9 wherein said methanol-containing fuel derives from captured CO₂.

11. The method of claim 10 which comprises alleviating global warming by capturing carbon dioxide from air and converting it into a methanol-containing fuel, and using said methanol-containing fuel to power motor vehicles, so as to yield substantially no net increase in atmospheric carbon dioxide from the burning of said fuel.

12. Apparatus of claim 6 which comprises: means for installing a cartridge loaded with a reversible CO₂-capturing agent in a motor vehicle and connecting it to the outlet of the vehicle's fuel combustion source so as to cause the effluent of said source to pass through said cartridge; means for removing a CO₂-enriched cartridge from said vehicle and replacing it by a fresh cartridge; means for treating said CO₂-enriched cartridge so as to yield a stream of concentrated carbon dioxide; means for feeding said stream of carbon dioxide into a methanol synthesis reactor; means for supplying methane and steam to said reactor; and wherein said means for reacting said methane and steam with said stream of carbon dioxide in said reactor so as to form a liquid fuel for powering a motor vehicle comprises a suitable catalyst and means for maintaining said catalyst at an appropriate temperature and pressure.

13. A method of converting captured carbon dioxide from motor vehicle exhausts and other fossil fuel burning sources which comprises the steps of feeding said carbon dioxide to a methanol synthesis reactor, maintaining said reactor at a suitable temperature and pressure, and reacting said carbon dioxide with methane and steam over a suitable catalyst so as to yield methanol or a methanol derivative.

14. The method of claim 9 wherein said carbon dioxide, methane, and steam are fed into said reactor in an approximate molar ratio of 1 to 3 to 2.

15. The method of claim 10 wherein said carbon dioxide, methane, and steam are fed into said reactor through appropriate control valves.

16. The method of claim 9 wherein said methanol or methanol derivative is fed from said reactor through appropriate valve controls to a methanol collector.

17. The method of claim 9 which comprises capturing carbon dioxide from a volume of an air mixture, passing said mixture through a removable cartridge loaded with a reversible CO₂-capturing agent; generating a concentrated stream of carbon dioxide by heating said reversible CO₂-capturing agent; and either sequestering said stream or converting it to a usable product, wherein said air mixture is an effluent of a motor vehicle exhaust, enclosing said CO₂-capturing agent within a removable cartridge connected to said motor vehicle exhaust; and providing a refueling station wherein a CO₂-enriched cartridge can be disconnected from said exhaust and replaced by a CO₂-depleted cartridge, and wherein the CO₂ from said enriched cartridge can be collected and converted into a methanol-containing fuel or other usable product.

18. The method of claim 9 wherein said effluent derives from the burning of a methanol-containing fuel.

19. The method of claim 10 wherein said methanol-containing fuel derives from captured CO₂.

20. The method of claim 11 which comprises alleviating global warming by capturing carbon dioxide from air and converting it into a methanol-containing fuel, and using said methanol-containing fuel to power motor vehicles, so as to yield no net increase in atmospheric carbon dioxide from the burning of said fuel.

21. Apparatus of claim 12 wherein said catalyst preferably contains a Cu—ZnO-alumina formulation preferably maintained at about 250-300 C and 50-100 atmospheres.
